(19) United States
(12) Patent Application Publication
(10) Pub. No.: US 2025/0256093 A1
(43) Pub. Date: Aug. 14, 2025

(54) ELECTRODE LEADS HAVING NERVE CUFFS WITH INFLATABLE NERVE CUFF STRAIGHTENERS

(71) Applicant: The Alfred E. Mann Foundation for Scientific Research, Valencia, CA (US)

(72) Inventor: Christopher Reed Jenney, Valencia, CA (US)

(73) Assignee: The Alfred E. Mann Foundation for Scientific Research, Valencia, CA (US)

(21) Appl. No.: 19/034,472

(22) Filed: Jan. 22, 2025

Related U.S. Application Data
(60) Provisional application No. 63/552,445, filed on Feb. 12, 2024.

(51) Int. Cl. A61N 1/05

(52) U.S. Cl. CPC A61N 1/0558; A61N 1/0556

(57) ABSTRACT

An electrode lead comprising an elongate lead body having a proximal end, a distal end and a plurality of electrical conductors, a biologically compatible, elastic, electrically insulative nerve cuff body that is associated with the distal end of the lead body, is configured to be circumferentially disposed around a nerve, has a pre-set furled state that defines an inner lumen and is movable to an unfurled state, a plurality of electrically conductive contacts carried by the nerve cuff body and electrically associated with the electrical conductors, and an inflatable nerve cuff straightener associated with the nerve cuff body and configured to at least substantially straighten the nerve cuff body when in a fully inflated state.

### CROSS-REFERENCE TO RELATED APPLICATION

This application claims the benefit of U.S. Provisional Application No. 63/552,445, filed Feb. 12, 2024, and entitled “Inflatable Nerve Cuffs,” which is incorporated herein by reference.

### BACKGROUND OF THE INVENTIONS

### 1. Field of Inventions

The present inventions relate generally to the treatment of obstructive sleep apnea by stimulating the hypoglossal nerve.

### 2. Description of the Related Art

Obstructive sleep apnea (OSA) is a highly prevalent sleep disorder that is caused by the collapse of or increase in the resistance of the pharyngeal airway, often resulting from tongue obstruction. The obstruction of the upper airway is mainly caused by reduced genioglossus muscle activity during the deeper states of non-rapid eye movement (NREM) sleep. In some OSA patients, obstruction occurs predominantly during rapid eye movement (REM) sleep. This is known as REM OSA and has different cardiometabolic and neurocognitive risks. Obstruction of the upper airway causes breathing to pause during sleep. Cessation of breathing, in turn, causes a decrease in the blood oxygen saturation level, which is eventually corrected when the person wakes up and resumes breathing. The long-term effects of OSA include, but are not limited to, high blood pressure, heart failure, strokes, diabetes, headaches, and general daytime sleepiness and memory loss.

Some proposed methods of alleviating apneic events involve the use of neurostimulators to open the upper airway. Such therapy involves stimulating the nerve fascicles of the hypoglossal nerve (HGN) that innervate the intrinsic and extrinsic muscles of the tongue in a manner that prevents retraction of the tongue, which would otherwise close the upper airway during the inspiration portion of the respiratory cycle. In some instances, the trunk of the HGN is stimulated with a nerve cuff, including a cuff body and a plurality of electrically conductive contacts (sometimes referred to as “electrodes”) on the cuff body, that is positioned around the HGN trunk. To that end, some nerve cuffs are pre-shaped to a furled state, may assume slightly less furled states, and may be unfurled to a flattened state. The HGN trunk nerve cuff may be configured in such a manner that it can be used to selectively stimulate nerve fascicles which innervate muscles that extend the tongue, while avoiding other nerve fascicles, with what is predominantly radial vector stimulation. HGN branches may also be stimulated. For example, an HGN GM branch may be stimulated with what is predominantly axial vector stimulation.

Exemplary nerve cuffs are illustrated and described in U.S. Pat. Pub. Nos. 2018/0318577A1, 2018/0318578A1, 2019/0060646A1, 2019/0282805A1, 2022/0062629A1, 2022/0313987A1, 2023/0010510A1, 2023/0241394A1, 2024/0009452A1, and 2024/0108883A1, which are incorporated herein by reference in their entirety.

### SUMMARY

The present inventors have determined that nerve cuffs are susceptible to improvement. In particular, at least some nerve cuffs are pre-set (or “pre-shaped”) to a furled (or “curled”) state that causes the nerve cuff to self-wrap around the associated nerve. The present inventors have determined that nerve cuffs which are pre-set to a furled state are advantageous because, for example, they do not require the physician to manually wrap the cuff around the nerve and do not require sutures, or specialty clips or clamps, to hold the cuff is place, as do flat nerve cuffs. The present inventors have, nevertheless, determined that conventional nerve cuffs which are pre-set to a furled state are susceptible to improvement. For example, the present inventors have determined that the process of straightening the nerve cuff so that it may be placed around the nerve can be difficult, can damage the nerve cuff, can make it difficult to accurately place the nerve cuff, and can increase the duration of the surgical procedure.

An electrode lead in accordance with at least one of the present inventions includes an elongate lead body having a proximal end, a distal end and a plurality of electrical conductors, a biologically compatible, elastic, electrically insulative nerve cuff body that is associated with the distal end of the lead body, is configured to be circumferentially disposed around a nerve, has a pre-set furled state that defines an inner lumen and is movable to an unfurled state, a plurality of electrically conductive contacts carried by the nerve cuff body and electrically associated with the electrical conductors, and an inflatable nerve cuff straightener associated with the nerve cuff body and configured to at least substantially straighten the nerve cuff body when in a fully inflated state. The present inventions also include systems with an implantable pulse generator or other implantable stimulation device in combination with such an electrode lead.

A method in accordance with at least one of the present inventions includes at least partially unfurling a nerve cuff, having a pre-set furled state that defines an inner lumen, by supplying pressurized fluid (i.e., liquid or gas) to the nerve cuff, positioning the at least partially unfurled nerve cuff adjacent to a nerve, and allowing the at least partially unfurled nerve cuff to return to the pre-set furled state, with the nerve located within the inner lumen, by removing at least some of the pressurized fluid from the nerve cuff.

There are several advantages associated with the present electrode leads and methods. Byway of example, but not limitation, the present electrode leads and methods simplify the process of straightening the nerve cuff so that it may be placed around the nerve. The physician can also selectively adjust the curvature of the nerve cuff by varying the pressure within the inflatable cuff straightener as the nerve cuff is being positioned around the HGN trunk or HGN GM branch. The nerve cuff will return to the pre-shaped furled state around the nerve when the pressurized fluid is removed therefrom. The ability to selectively unstraighten portions of the nerve cuff and adjust the curvature with the movable cuff straightener reduces the difficulty, duration and likelihood of cuff damage associated with the implantation process.

### BRIEF DESCRIPTION OF THE DRAWINGS

Detailed descriptions of exemplary embodiments will be made with reference to the accompanying drawings.

FIG. 1 is a plan view of a stimulation system including an electrode lead in accordance with one embodiment of a present invention.

FIG. 2 is a side view of a portion of the stimulation system illustrated in FIG. 1.

FIG. 3 is a side view showing the nerve cuff illustrated in FIG. 1 in a pre-shaped furled and uninflated state.

FIG. 4 is a front view of the nerve cuff illustrated in FIG. 1 in an unfurled and inflated state.

FIG. 5 is a rear view of the nerve cuff illustrated in FIG. 1 in an unfurled and inflated state.

FIG. 6 is a cut-away anatomical drawing of the head and neck area illustrating the muscles that control movement of the tongue, the HGN and its branches that innervate these muscles, and the nerve cuff illustrated in FIG. 1 on the HGN trunk.

FIG. 7 is a side view showing the nerve cuff illustrated in FIG. 1 in a pre-shaped furled state around an HGN branch.

FIG. 8 is a side view of the nerve cuff illustrated in FIG. 1 in an unfurled and inflated state.

FIG. 8A is a side view of a nerve cuff in accordance with one embodiment of a present invention in an unfurled and inflated state.

FIG. 9 is a flow chart showing a method in accordance with one embodiment of a present invention.

FIG. 10 is a front view of the nerve cuff illustrated in FIG. 1 in an unfurled and inflated state.

FIG. 11 is a rear, cutaway view of the nerve cuff illustrated in FIG. 1 in an unfurled and inflated state.

FIG. 12 is a rear view of the nerve cuff illustrated in FIG. 1 in an unfurled and inflated state.

FIG. 13 is a section view taken along line 13-13 in FIG. 12.

FIG. 14 is a section view taken along line 14-14 in FIG. 3.

FIG. 15 is a section view taken along line 15-15 in FIG. 12.

FIG. 16 is a front view of a nerve cuff in accordance with one embodiment of a present invention in an unfurled and inflated state.

FIG. 17 is a rear, cutaway view of the nerve cuff illustrated in FIG. 16 in an unfurled and inflated state.

FIG. 18 is a rear view of a nerve cuff in accordance with one embodiment of a present invention in an unfurled and inflated state.

FIG. 19 is a rear view of a nerve cuff in accordance with one embodiment of a present invention in an unfurled and inflated state.

FIG. 20 is a rear view of a nerve cuff in accordance with one embodiment of a present invention in an unfurled and inflated state.

FIG. 21 is a side view showing a nerve cuff in accordance with one embodiment of a present invention in a pre-shaped furled and uninflated state.

FIG. 22 is a section view taken along line 22-22 in FIG. 21.

FIG. 23 is a rear view of the nerve cuff illustrated in FIG. 21 in an unfurled and inflated state.

FIG. 24 is a section view taken along line 24-24 in FIG. 23.

### DETAILED DESCRIPTION OF THE EXEMPLARY EMBODIMENTS

The following is a detailed description of the best presently known modes of carrying out the inventions. This description is not to be taken in a limiting sense but is made merely for the purpose of illustrating the general principles of the inventions.

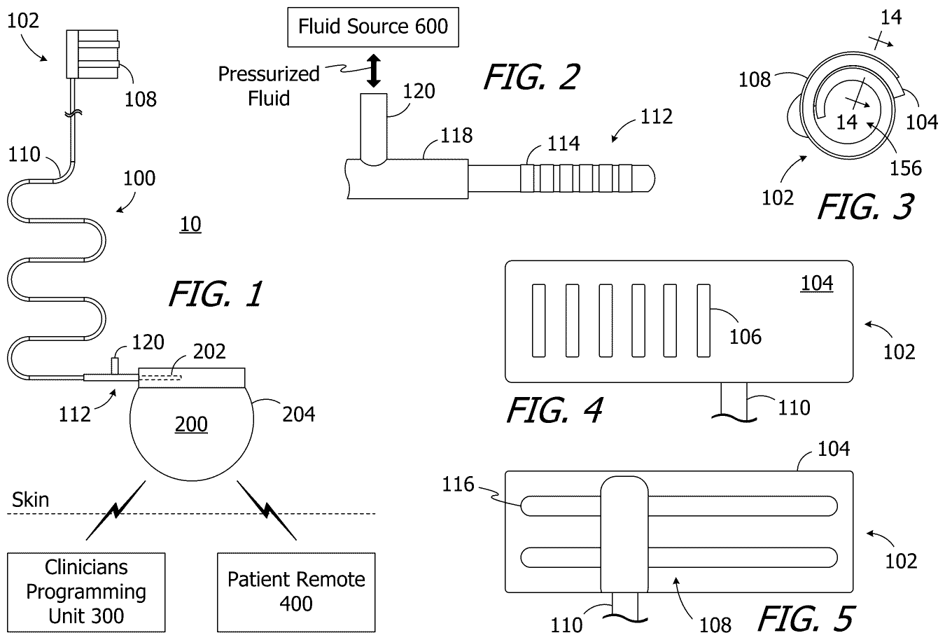

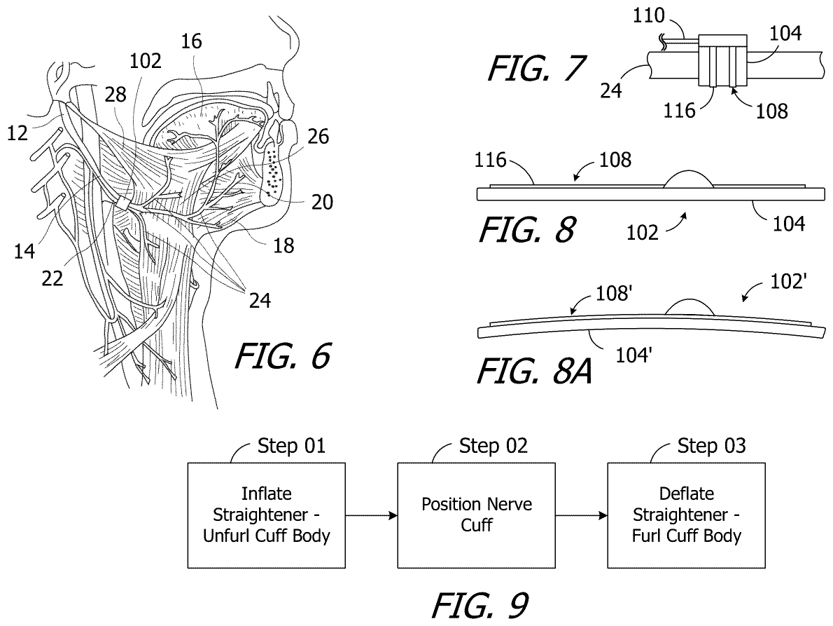

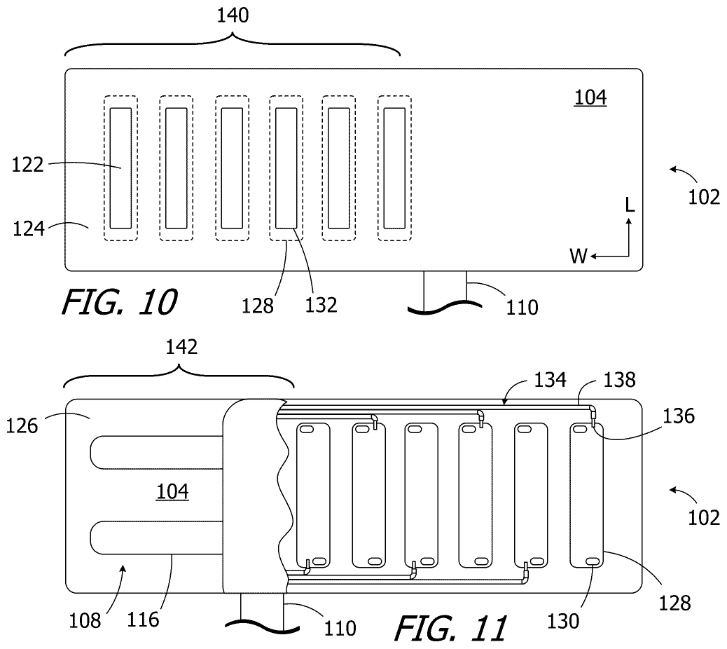

Referring to FIGS. 1-5, a stimulation system 10 in accordance with one embodiment of a present invention includes an electrode lead 100 and an implantable stimulator such as the implantable pulse generator (“IPG”) 200. A clinician's programming unit 300, a patient remote 400 and an IPG charger (not shown) may also be provided in some instances. The exemplary electrode lead 100 includes a nerve cuff 102, with a cuff body 104, electrically conductive contacts (or “electrodes”) 106 and an inflatable cuff straightener 108, and a lead body 110. The lead body 110 couples the nerve cuff 102 to the IPG 200 by way of a lead connector 112 with contacts 114 on the proximal end of the lead body 110 and a corresponding connector receptacle 202 on the IPG 200. The exemplary cuff body 104 and electrically conductive contacts 106 are discussed in greater detail below with reference to FIGS. 10 and 11. The inflatable cuff straightener 108 may be used to straighten cuff body 104 (and the nerve cuff 102) to facilitate placement onto and around a nerve. For example, the cuff body 104 (and nerve cuff 102) may be pre-set (or “pre-shaped”) to the furled (or “curled”) state illustrated in FIG. 3, where the inflatable cuff straightener 108 is in an uninflated state, which allows the cuff body 104 to be circumferentially disposed around either the HGN trunk or a HGN branch (e.g., the HGN GM branch), as is described below with reference to FIGS. 6 and 7. The inflatable cuff straightener 108 may be inflated with a pressurized fluid to such an extent that the inflatable cuff straightener 108 straightens, thereby overcoming the stiffness of the cuff body 104 and straightening the cuff 102 in the manner illustrated in FIGS. 4 and 5.

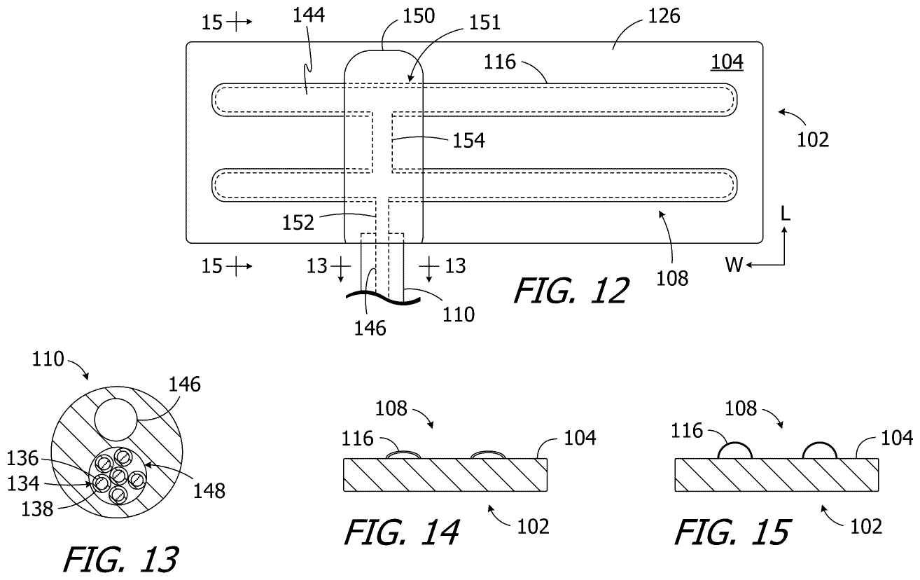

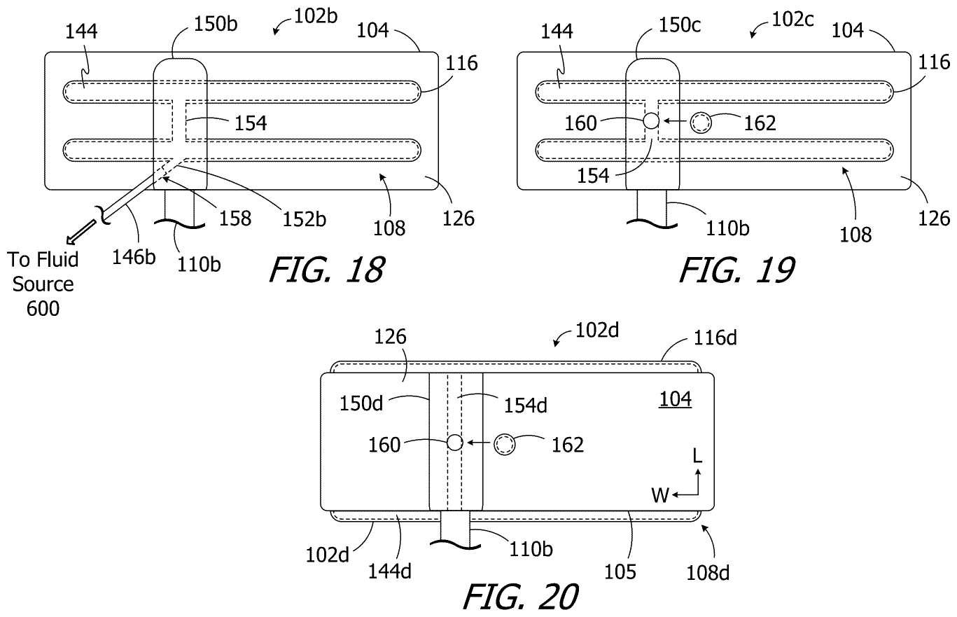

The exemplary inflatable cuff straightener 108 includes two inflatable members 116, although the number of inflatable members may be increased or decreased. Any suitable pressurized fluid (liquid or gas) may be supplied to and used to inflate the inflatable members 116, which are described in greater detail below with reference to FIGS. 12-15. The pressurized fluid may be supplied by a fluid source 600, as is described below. To that end, in some implementations, the lead connector 112 includes a grip zone 118 with a fluid port 120. The fluid port 120, which is configured to be connected to the fluid source 600, is in fluid communication with a fluid lumen 146 (FIG. 13) within the lead body 110 that extends to the nerve cuff where it is fluidically coupled to the inflatable members 116. Fluid may be supplied to inflatable cuff straighteners in other ways, as is discussed below with reference to FIGS. 18 and 19.

The exemplary lead body 110 may also include one or more S-shaped sections (FIG. 1) to provide strain relief (as shown) or may be straight. The S-shaped sections accommodate body movement at the location within the neck where the lead body 110 is implanted, thereby reducing the likelihood that the HGN will be damaged due to unavoidable pulling of the electrode lead 100 that may result from neck movements. The accommodation provided by the S-shaped sections also reduces the likelihood of fatigue damage. Additionally, although the exemplary system 10 includes a single electrode lead 100, other embodiments may include a pair of electrode leads 100 for bilateral HGN stimulation and an IPG (not shown) with two connector receptacles.

Turning to FIG. 6, and as alluded to above, the nerve cuff 102 may be positioned around the trunk 14 of the HGN 12 and used to stimulate the muscles that anteriorly move the tongue 16 and, in particular, the fascicles of the HGN 12 that innervate the tongue protrusor muscles, such as the genioglossus 18 and/or the geniohyoid muscles 20. The nerve cuff 102 is positioned on the HGN trunk 14 at a position 22 proximal to the HGN branches 24. Although there are advantages to implanting the nerve cuff 102 at this proximal position 22, i.e., reduced surgical time and effort as well as reduced risk and trauma to the patient, it introduces the problem of inadvertently stimulating other fascicles of the HGN trunk 14 that innervate muscles in opposition to the genioglossus 18 and/or the geniohyoid muscles 20, i.e., the tongue retractor muscles, e.g., the hyoglossus 26 and styloglossus muscles 28, as well as the intrinsic muscles of the tongue 16. Accordingly, while some clinicians may desire to stimulate the HGN 12 at the HGN trunk 14, others may desire to stimulate the HGN at the GM branch 24. As illustrated in FIG. 7, the nerve cuff 102 is configured in such a manner that it may be positioned the HGN GM branch 24 as well as the trunk 14 in various furled states. In the illustrated implementation, the nerve cuff 102 is pre-set (or “pre-shaped”) to the furled (or “curled”) state illustrated in FIG. 7.

In one exemplary method of using the inflatable cuff straightener 108 to facilitate positioning of the nerve cuff 102 of the electrode lead 100 (as well as the other inflatable cuff straighteners and nerves cuffs described herein) around the HGN trunk 14 or HGN GM branch 24, pressurized fluid is supplied by a fluid source 600 to the inflatable members 116. Full pressurization will result in the nerve cuff transitioning from the furled state to an at least substantially straightened state, i.e., either a straightened state or slightly curved state depending on the characteristics of the various aspects of the nerve cuff and pressure of the supplied fluid. The respective characteristics of the cuff body 104 and the inflatable cuff straightener 108 result in the nerve cuff 102 transitioning to a straightened state illustrated in FIG. 8 at full pressurization. In other instances, the respective characteristics of the cuff body 104′ and the inflatable cuff straightener 108′ result in the nerve cuff 102′ transitioning to a slightly curved, at least substantially straightened state such as that illustrated in FIG. 8A at full pressure.

One exemplary method of positioning the present nerve cuffs is summarized in the flow chart illustrated in FIG. 9 and is described in the exemplary context of nerve cuff 102. The illustrated method is also applicable to the other nerve cuffs described herein. First, the inflatable members 116 of the cuff straightener 108 are inflated to unfurl and at least substantially straighten the cuff body 104 (Step 01). The inflation may be performed at the time of the implantation surgery or at any other appropriate time. The pressure may be modulated as the nerve cuff is being positioned around the HGN trunk or HGN GM branch (Step 02) to selectively adjust the curvature of the cuff body 104 (and nerve cuff 102). Removal of the fluid, e.g., by applying suction with the fluid source 600 or by allowing the structural biasing force that pre-shapes the cuff body 104 to drive the fluid out, deflates the inflatable members 116 (Step 03) and returns the cuff body 104 to a furled state, such as that illustrated in FIGS. 3, 6 and 7, with the nerve cuff 102 wrapped around the HGN trunk or HGN GM branch. The fluid source 600 may also be disconnected from the nerve cuff after depressurization.

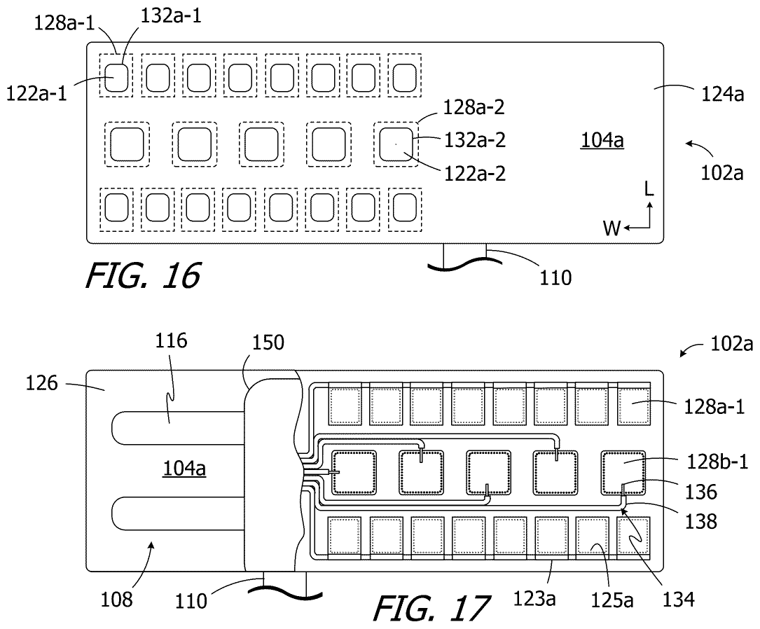

The present electrode leads may include any suitable nerve cuff configuration. As illustrated for example in FIGS. 10 and 11, the exemplary nerve cuff 102 includes the cuff body 104, which defines a length L and a width W that is greater than the length in the unfurled state, and a plurality of electrically conductive contacts (or “contacts”) 122 on the cuff body 104. Such contacts may also be referred to as “electrodes.” Although the present inventions are not so limited, the contacts 122 are relatively narrow contacts, i.e., are contacts with a greater length than width, and are spaced from one another in the width direction. Although the number may increase or decrease in the context of other nerve applications, there are six contacts 122 in the illustrated embodiment. With respect to shape, and although the present inventions are not so limited, the contacts 122 are the same shape, i.e., rectangles with rounded corners. The contacts 122 are also the same size. In other implementations, the contacts within a particular nerve cuff may differ in shape, size, and/or orientation. Other exemplary contact shapes include, but are not limited to, rounded rectangles, circles, ovals, and squares. Some or all of the contacts may also be relatively wide, as is discussed below with reference to FIGS. 16 and 17.

The cuff body 104 and contacts 122 may be of any suitable construction. In the illustrated implementation, the cuff body 104 includes a front layer 124 that will face the HGN trunk or branch (and defines the front surface) and a rear layer 126 that will face away from the HGN trunk or branch (and defines the rear surface). Conductive members 128 are located between the front layer 124 and rear layer 126 and may also include apertures 130 that, in conjunction with the material that forms the front and rear layers and enters the apertures, anchor the conductive members in their intended locations. The conductive members 128 are each exposed by way of respective openings 132 in the cuff body front layer 124. The openings 132 are located inwardly of the outer perimeter of the conductive members 128, which are shown in dashed lines in FIG. 10. Referring to FIG. 11, the contacts 122 in the illustrated embodiment may be individually electrically connected to the plurality contacts 114 on the lead connector 112 (FIG. 2) by wires 134 that extend through the lead body 110. Each wire 134 includes a conductor 136 and an insulator 138. The conductors 136 may be connected to the rear side of the conductive members 128 by welding or other suitable processes. The cables or other electrical conductors may be employed in place of the wires 134 in other implementations.

With respect to the location of the contacts 122 in the exemplary nerve cuff 102, the cuff body 104 includes a stimulation region 140 and a compression region 142. The contacts 122 are located within the stimulation region 140. There are no contacts located within the compression region 142. The compression region 142 wraps around at least a portion of the stimulation region 140 when the nerve cuff 102 is in the pre-shaped furled state and slightly larger, expanded and less tightly furled states, thereby resisting (but not preventing) expansion of the stimulation region and improving the electrical connection between the contacts 122 and the HGN.

Turning to FIGS. 12-15, and as alluded to above, the exemplary inflatable cuff straightener 108 includes two inflatable members 116 that are located on the rear surface of the cuff body 104. The inflatable members 116 define internal pressure chambers 144 that are connected to the fluid source 600 by way of the exemplary lead body 110. To that end, the exemplary lead body 110 may include an internal fluid lumen 146 and an internal wire lumen 148, and the nerve cuff 102 may include a manifold 150 that is secured to rear layer 126 of the cuff body 104, with lumens 152 and 154, to anchor the lead body 110 to the rear layer 126 of the cuff body 104. Lumen 152 fluidically connects the fluid lumen 146 to one of the pressure chambers 144, and lumen 154 connects the pressure chambers 144 to one another. The manifold 150 also functions as a strain relief and provides passage for the wires 134.

In the illustrated implementation, the manifold 150 defines passages 151 that the inflatable members 116 extend through. The size and shape of the passages corresponds to the size and shape of the inflatable members 116 in their deflated states. To that end, it should be noted that the exemplary inflatable members 116 may be elastomeric and expand linearly with pressure when the inflated, or may simply be flexible but not elastomeric. An elastomeric inflatable member 116 may in some instances bulge, and slightly re-furl the nerve cuff 102, if it is inflated to such an extent that it elongates. A non-elastomeric inflatable member 116 will not bulge if overinflated. It will simply straighten the cuff and will collapse to a relatively thin or small profile when deflated.

The inflatable members 116 of the exemplary cuff straightener 108, which are shown in a deflated state in FIGS. 3 and 14 and in an inflated state in FIGS. 12 and 15, may be separately formed structures that are secured to the rear side of the cuff body 104 or may be an integral part of the rear layer 126. Other configurations are described below with reference to FIGS. 20-24. In any case, the configuration of the inflatable members 116 and the degree of pressurization provided by the fluid source 600 are sufficient to hold cuff body in the unfurled state illustrated in in FIGS. 12 and 15.

In the illustrated implementation, the inflatable members 116 (and inflatable cuff straightener 108) define a length and a width that is greater than the length. The width of the inflatable members 116 (and inflatable cuff straightener 108) is slightly less, i.e., about 10% less, than the width of the cuff body in the embodiment illustrated in FIG. 12. The width of the inflatable members 116 (and inflatable cuff straightener 108) may range from about 60% to about 100% of the width of the cuff body 104. As used herein in the context of percentages, the word “about” means±10-20%, although the width of the inflatable members 116 (and inflatable cuff straightener 108) will typically not be greater than the width of the cuff body 104.

The pressurized fluid supplied by the fluid source 600 may be a gaseous fluid, such as air or helium, a liquid fluid such as water or saline, any other sufficiently compliant media, and combinations thereof. The fluid source 600 may be configured to create both positive and negative fluid pressures in order to inflate and deflate the inflatable members 116 of the inflatable cuff straightener 108. Suitable fluid sources include, but are not limited to, pumps and syringes, and may include a coupling (not shown) that is configured to connect to the fluid port 120 (FIGS. 1 and 2).

In some instances, one of more valves (not shown) that regulated the flow of pressurized fluid, prevent overinflation, and/or prevent pressurized fluid from escaping prematurely may be provided. The valve(s) may, for example, be associated with the fluid port 120 (FIG. 2), the port 160 (FIGS. 19 and 20), and/or the fluid source 600 (FIG. 2).

With respect to materials and dimensions, the exemplary cuff body 104 may be formed from any suitable material. Such materials may be biologically compatible, electrically insulative, elastic and capable of functioning in the manner described herein. By way of example, but not limitation, suitable cuff body materials include silicone, polyurethane and styrene-isobutylen-styrene (SIBS) elastomers. Suitable materials for the contacts 122 include, but are not limited to, platinum-iridium and palladium. The cuff materials should be pliable enough to allow inflatable cuff straightener 108 or a clinician to hold the cuff body 104 (and nerve cuff 102) in an unfurled state when the nerve cuff 102 is being placed around the HGN trunk (or HGN GM branch). The exemplary materials are also resilient enough to cause the nerve cuff return 102 to the pre-shaped furled state illustrated in FIG. 3 when the inflatable members 116 are deflated, yet flexible enough to allow the cuff body 104 (and nerve cuff 102) to instead assume the slightly larger, expanded and less tightly furled, furled states. For example, the inner lumen 156 defined by the cuff body 104 (and nerve cuff 102) in FIG. 3 is sized to accommodate an HGN structure that has a diameter of about 2.5 mm (e.g., the HGN GM branch 24). The cuff body 104 (and nerve cuff 102) is also cable of assuming less furled states that are less tightly furled to, for example, accommodate an HGN structure that has a diameter of about 3.0 mm (e.g., the HGN GM branch 24 in a swollen state) and an HGN structure that has a diameter of about 4.0 mm (e.g., the HGN trunk 14). The ability to assume slightly larger, expanded and less tightly furled states, in addition to the smaller fully furled state, allows the same nerve Cuff 102 to accommodate either of the larger HGN trunk 14 or a smaller HGN branch 24. The ability to assume slightly larger, expanded furled states also allows the nerve cuff to accommodate nerve swelling that may occur post-surgery and to self-adjust to a smaller state when the swelling subsides. It should also be noted here that the width of the stimulation region 140 is such that it extends completely around the inner lumen 156, i.e., 360° or more around the longitudinal axis of the inner lumen, when the cuff body 104 is in the fully furled state illustrated in FIG. 3 that accommodates an HGN structure having a diameter of about 2.5 mm. The stimulation region 140 also extends substantially around the inner lumen 156, i.e., at least 288° in some examples and 360° or more in other examples, when the cuff body 104 is in an expanded and less tightly furled state that accommodates an HGN structure having a diameter of about 4.0 mm. The dimensions of the present nerve cuffs, including the various elements thereof, may by any dimensions that result in the nerve cuffs functioning as intended. With respect to the dimensions of the cuff body 104 of the exemplary nerve cuff 104, and referring to FIGS. 10 and 11, the cuff body is about 1.1 inches wide and about 0.34 inches long. As used herein in the context of dimensions, the word “about” means±10-20%. The width of the stimulation region 140 is about 0.6 inches, while the width of the compression region 142 is about 0.5 inches.

Turning to the inflatable members 116, the inflatable members may be formed from any suitable material that can accommodate elastic pressurization during inflation. Pliable, elastic, and biocompatible materials such as silicone, nylon, Urethane, vinyl, latex rubber, PET and polyolefins or combinations thereof, may be employed. Non-biocompatible materials may be coated with a layer of biocompatible material and sufficiently sealed within the interior of the cuff body.

The embodiments described above are susceptible to a wide variety of modifications. For example, the present inventions are not limited to any particular cuff body or contact configuration. Nerve cuffs with helical cuff bodies and/or nerve cuffs with coil contacts may include any of the inflatable cuff straighteners described herein. One exemplary alternative contact configuration is illustrated in FIGS. 16 and 17 in the context of exemplary nerve cuff 102a. Nerve cuff 102a is substantially similar to nerve cuff 102 and similar elements are represented by similar reference numerals. Here, however, the nerve cuff 102a includes first and second pluralities of electrically conductive contacts 122a-1 on the cuff body 104a, and a plurality of electrically conductive contacts 122a-2 between the first and second pluralities of electrically conductive contacts 122a-1. The contacts 122a-1 in are spaced from one another in the width direction, as are the contacts 122a-2, and the contacts may also be referred to as “electrodes.” The contacts 122a-1 within each plurality are connected to one another in series and function as a single relatively wide contact. To that end, the contacts 122a-1 within each plurality are connected to a single wire 134 with crimp regions 123a. The contacts 122a-2 are not connected to one another in series and are instead each connected to a separate wire 134. Additionally, as compared to the each of the pluralities of contacts 122a-1, the contacts 122a-2 are relatively narrow. The contacts 122a-1 and 122a-2 are formed by conductive members 128a-1 and 128a-2, which are located between the front layer 124a and the rear layer 126 and are exposed by way of windows 132a-1 and 132a-2 in the cuff body front layer 124a. In some instances, the conductive members 128a-1 and 128a-2 may include a plurality of holes 125a that extend completely through the conductive members 128a-1 and 128a-2 and through which the cuff body material extends, thereby forming anchors that reduce the likelihood of delamination and/or conductive member dislodgement.

Other exemplary cuff body and contact configurations that may be employed in either instance include, but are not limited to, those illustrated and described in U.S. Pat. Pub. Nos. 2018/0318577A1, 2018/0318578A1, 2019/0060646A1, 2019/0282805A1, 2022/0062629A1, 2022/0313987A1, 2023/0010510A1, 2023/0241394A1, 2024/0009452A1, and 2024/0108883A1.

Turning to non-limiting examples of other inflatable cuff straighteners, the exemplary nerve cuff 102b illustrated in FIG. 18 is substantially similar to nerve cuff 102 and similar elements are represented by similar reference numerals. Here, however, the lead body 110b does not include a fluid lumen and the manifold 150b is connected to the fluid source 600 by way of a separate fluid tube 146b. The exemplary fluid tube 146b is permanently connected to the manifold lumen 152b at a port 158. After the inflatable cuff straightener 108 has been inflated and deflated and the nerve cuff 102b is positioned on a nerve, the fluid tube 146b may be severed and in some instances sealed (e.g., with a clip or heat). Alternatively, as shown in FIG. 19, the nerve cuff 102c includes a manifold 150c with a port 160 that is configured to receive a coupling (not shown) at the end of a tube that is connected to the fluid source 600. A plug 162 may be used to seal the port 160.

The location of the inflatable cuff straightener is not limited to the rear side of the cuff body. By way of example, but not limitation, inflatable members may be located on the side edges of a cuff body. To that end, and referring to FIG. 20, the exemplary nerve cuff 102d is substantially similar to nerve cuff 102 and similar elements are represented by similar reference numerals. Here, however, the inflatable cuff straightener 108d includes a pair of inflatable members 116d, with internal pressure chambers 144d, that extend in the width direction along the side edges 105 of the cuff body 104. The inflatable members 116d may be only on the side edges 105 (as shown), or may have small portions than extend onto the front and/or rear surfaces 124 and 126 of the cuff body 104. The nerve cuff 102d also includes a manifold 150d with a lumen 154d that is fluidically connected to both of the pressure chambers 144d. Pressurized fluid may pass in and out of the manifold 150d by way of a port 160 that is configured to receive a coupling (not shown) at the end of a tube that is connected to the fluid source 600. Here too, a plug 162 may be used to seal the port 160.

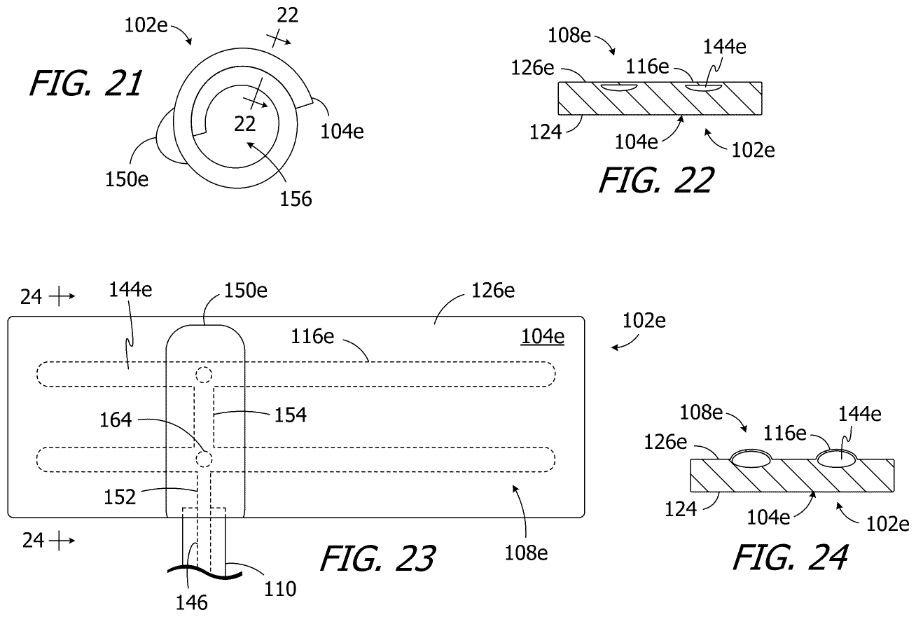

Inflatable cuff straighteners may also be located within nerve cuff bodies. For example, the nerve cuff 102e illustrated in FIGS. 21-24 is substantially similar to nerve cuff 102 and similar elements are represented by similar reference numerals. Here, however, the inflatable cuff straightener 108e is located within, and is formed by, portions of the cuff body 104e. In particular, the exemplary cuff straightener 108e includes a pair of inflatable members 116e, with internal pressure chambers 144e, that are located under the rear surface (i.e., are located between the front and rear surfaces) and are formed at least in part by the rear layer 126e. The internal pressure chambers 144e are connected to the lumen 154 within manifold 150e by apertures 164. Supplying pressurized fluid to the pressure chambers 144e (e.g., with the fluid source 600), thereby pressurizing and straightening the inflatable members 116e, distends and straightens the inflatable members 116e, thereby driving the cuff body 104e from the furled state illustrated in FIGS. 21 and 22 to the unfurled state illustrated in FIGS. 23 and 24. The cuff body 104e will return to the furled state when the pressurized fluid is removed.

Although the inventions disclosed herein have been described in terms of the preferred embodiments above, numerous modifications and/or additions to the above-described preferred embodiments would be readily apparent to one skilled in the art.

By way of example, although the exemplary cuff straighteners described above include two fluidically connected inflatable members, the present inventions are not so limited. Other embodiments may include a single inflatable member, more than two inflatable members, and/or multiple inflatable members that are fluidically isolated from one another and are separately inflatable.

It is intended that the scope of the present inventions extend to all such modifications and/or additions. The inventions include any and all combinations of the elements from the various embodiments disclosed in the specification. The scope of the present inventions is limited solely by the claims set forth below.

1. An electrode lead, comprising:
an elongate lead body having a proximal end, a distal end and a plurality of electrical conductors;
a biologically compatible, elastic, electrically insulative nerve cuff body that is associated with the distal end of the lead body, is configured to be circumferentially disposed around a nerve, has a pre-set furled state that defines an inner lumen and is movable to an unfurled state;
a plurality of electrically conductive contacts carried by the nerve cuff body and electrically associated with the electrical conductors; and
an inflatable nerve cuff straightener associated with the nerve cuff body and configured to at least substantially straighten the nerve cuff body when in a fully inflated state.

2. The electrode lead claimed in claim 1, wherein
the nerve cuff body defines a front surface and a rear surface; and
the inflatable nerve cuff straightener is located on the rear surface.

3. The electrode lead claimed in claim 1, wherein
the nerve cuff body defines a front surface and a rear surface; and
the inflatable nerve cuff straightener is located between the front surface and the rear surface.

4. The electrode lead claimed in claim 1, wherein
the nerve cuff body defines a front surface, a rear surface and side surfaces; and
the inflatable nerve cuff straightener is located on at least one of the side surfaces.

5. The electrode lead claimed in claim 1, wherein
the elongate lead body includes a fluid lumen that is in fluid communication with the inflatable nerve cuff straightener.

6. The electrode lead claimed in claim 1, wherein
the elongate lead body does not include a fluid lumen.

7. The electrode lead claimed in claim 5, further comprising:
a manifold on the nerve cuff body that is in fluid communication with the inflatable nerve cuff straightener.

8. The electrode lead claimed claim 1, wherein,
inflatable nerve cuff straightener comprises at least one inflatable member.

9. The electrode lead claimed in claim 8, wherein,
inflatable nerve cuff straightener comprises a plurality of inflatable members.

10. The electrode lead claimed in claim 1, wherein,
the nerve cuff body defines a length, a width in the unfurled state that is greater than the length, and a width direction; and
the inflatable nerve cuff straightener defines a length and a width in the unfurled state that is greater than the length and that is at least 60% of the width of the nerve cuff body.

11. The electrode lead claimed in claim 1, wherein,
the nerve cuff body defines a length, a width in the unfurled state that is greater than the length, and a width direction; and
the electrically conductive contacts each define a length and a width that is less than the length;
the electrically conductive contacts are spaced from one another in the width direction of the nerve cuff body.

12. The electrode lead claimed in claim 1, wherein,
the nerve cuff body defines a length, a length direction, a width in the unfurled state that is greater than the length, and a width direction; and
the electrically conductive contacts comprise first and second relatively wide electrically conductive contacts that are spaced from one another in the length direction and extend in the width direction to such an extent that they extend completely around the cuff body inner lumen when the cuff body is in the pre-set furled state and a plurality of relatively narrow electrically conductive contacts that are spaced from one another in the width direction and are located between the first and second relatively wide electrically conductive contacts.

13. A method, comprising:
at least partially unfurling a nerve cuff, having a pre-set furled state that defines an inner lumen, by supplying pressurized fluid to the nerve cuff;
positioning the at least partially unfurled nerve cuff adjacent to a nerve; and
allowing the at least partially unfurled nerve cuff to return to the pre-set furled state, with the nerve located within the inner lumen, by removing at least some of the pressurized fluid from the nerve cuff.

14. The method according to claim 13, wherein
the nerve cuff includes a nerve cuff body, a plurality of electrically conductive contacts carried by the nerve cuff body, and an inflatable nerve cuff straightener associated with the nerve cuff body and configured to at least substantially straighten the nerve cuff body when in a fully inflated state in response to receipt of the pressurized fluid.

15. The method according to claim 14, wherein
the nerve cuff is connected to an elongate lead body having a proximal end, a distal end a plurality of electrical conductors electrically associated with the electrically conductive contacts;
the pressurized fluid is supplied to the nerve cuff by way of the elongate lead body.

16. The method according to claim 14, wherein
the nerve cuff is connected to an elongate lead body having a proximal end, a distal end a plurality of electrical conductors electrically associated with the electrically conductive contacts;
the pressurized fluid is not supplied to the nerve cuff by way of the elongate lead body.

17. The method according to claim 13, wherein
the pressurized fluid is supplied by a fluid source.

18. The method according to claim 17, wherein
the pressurized fluid is removed with the fluid source.

19. The method according to claim 13, further comprising:
varying the fluid pressure within the nerve cuff while positioning the nerve cuff adjacent to the nerve and/or allowing the nerve cuff to return to the pre-set furled state,

20. The method according to claim 13, wherein
at least partially unfurling a nerve cuff comprises fully unfurling the nerve cuff.

21. The method according to claim 20, wherein
the fully unfurled nerve cuff is in an at least substantially straightened state.
